(19) United States
(12) Patent Application Publication
(10) Pub. No.: US 2013/0102967 A1
(43) Pub. Date: Apr. 25, 2013

(54) Magnetic Trocar System

(71) Applicants: Matthew A. Hanlon, O'Fallon, MO (US); Matthew LaConte, Maryland Heights, MO (US)

(72) Inventors: Matthew A. Hanlon, O'Fallon, MO (US); Matthew LaConte, Maryland Heights, MO (US)

(73) Assignee: SYNERGETICS, INC., O'Fallon, MO (US)

(21) Appl. No.: 13/278,730

(22) Filed: Oct. 21, 2011

(51) Int. Cl. A61B 17/34

(52) U.S. Cl. 604/170.01; 604/164.01

(57) ABSTRACT

Systems and methods for using a trocar system are disclosed. The system generally comprises a trocar, a magnet, and a magnetizable cannula. The trocar has a first end and a second end. The magnetizable cannula has a central opening sized to receive the trocar. The magnet is positioned adjacent the second end of the trocar and its magnetic field releasably secures the magnetizable cannula in a position adjacent the second end of the trocar.

### FIELD

This disclosure generally relates to trocar systems and, more specifically, to trocar systems that use a magnet to releasably secure a cannula to a trocar.

### BACKGROUND

Trocar systems are used to make an incision in a patient's body (e.g., in the eye) in a variety of surgical procedures. Typically, a cannula positioned around the trocar is inserted into the incision to keep the incision from closing after the trocar is removed. Other surgical implements may then be inserted through the cannula into the patient's body after removal of the trocar.

Trocar systems with pre-mounted cannulas typically include a mechanism to retain the cannula on the trocar to prevent the cannula from falling off the trocar prematurely. Previous systems have retained the cannula on the trocar using a variety of techniques. In one such system, a portion of the trocar is bent or deformed such that it has a cross-sectional dimension that is slightly larger than the diameter of a central opening of the cannula. Other systems retain the trocar on the cannula by using a trocar that has a cross-sectional dimension that is equal to or slightly smaller than the diameter of the trocar to create an interference fit between the two. Still other systems rely on friction between the trocar and the cannula caused by dissimilarities in cross-sectional shapes and flexible detents formed into the cannula.

These previous systems are not always reliable in retaining the cannula to the trocar or in establishing a consistent detachment force when attempting to withdraw the trocar after insertion of the cannula. Accordingly, an improved trocar system is needed.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

One aspect is a trocar system comprising a trocar, a magnet, and a magnetizable cannula. The trocar has a first end distal from a second end. The magnet is positioned adjacent second end of the trocar and the magnet generates a magnetic field. The magnetizable cannula has a central opening sized to receive the trocar. The magnetic field of the magnet releasably secures the cannula in a position adjacent the second end of the trocar.

Another aspect is a trocar system comprising a trocar, a handle, and a cannula. The trocar has a first end distal from a second end. The handle has a first end and a second end and the second of the trocar is connected to the second end of the handle. The cannula has a central opening sized to receive the trocar. The cannula is releasably secured in a position adjacent the second end of the trocar by a magnetic field.

Still another aspect is a method of using a trocar system. The trocar system comprises a trocar having a first end and a second end, a magnet, and a magnetizable cannula. The magnetizable cannula is releasably secured in a position adjacent the second end of the trocar by a magnetic field of the magnet. The method comprises the steps of inserting the first end of the trocar into an incision formed in a patient's body, inserting the magnetizable cannula into the incision in the patient's body, and removing the trocar from the magnetizable cannula.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a perspective of one embodiment of a trocar system with a cannula releasably secured in a position adjacent a handle;

FIG. 2 is a side view of the system of FIG. 1;

FIG. 3 is an enlarged perspective view of a cannula of FIG. 1;

FIG. 4 is an end view of the system of FIG. 1 with the cannula omitted for clarity; and

FIG. 5 is a perspective view of the trocar system of FIG. 1 with the cannula positioned adjacent a second end of the trocar.

Like reference symbols in the various drawings indicate like elements.

### DETAILED DESCRIPTION

The embodiments described herein generally relate to trocar systems that use a magnet to releasably secure a cannula to a trocar. Such systems may be used in various surgical procedures such as ophthalmic procedures, and in particular, intra-ocular ophthalmic procedures for vitreo-retinal surgery. Thus, while reference is made herein to use of the system in ophthalmic procedures, it should be understood that the system can also be used in other surgical procedures.

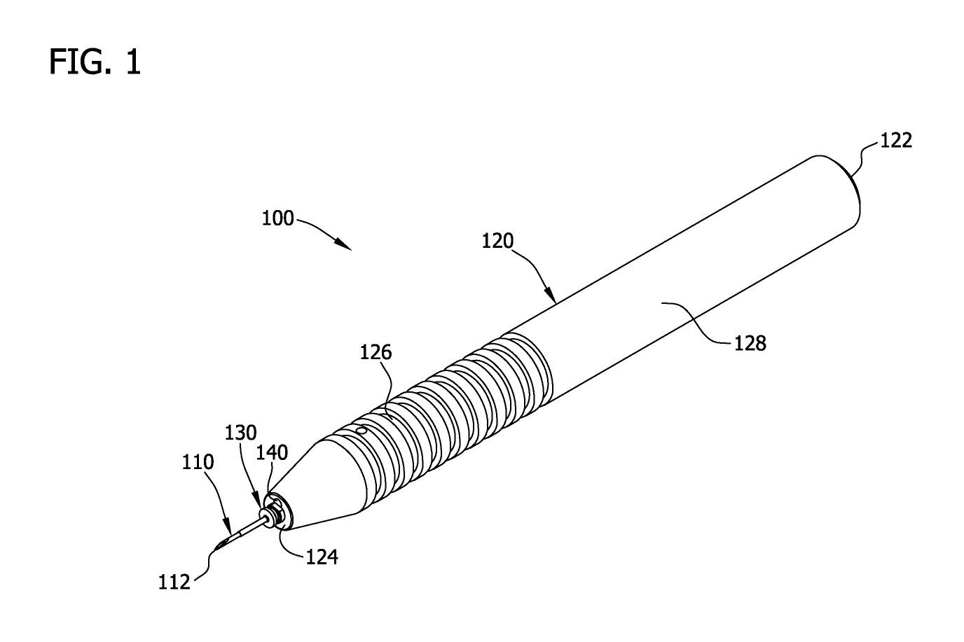

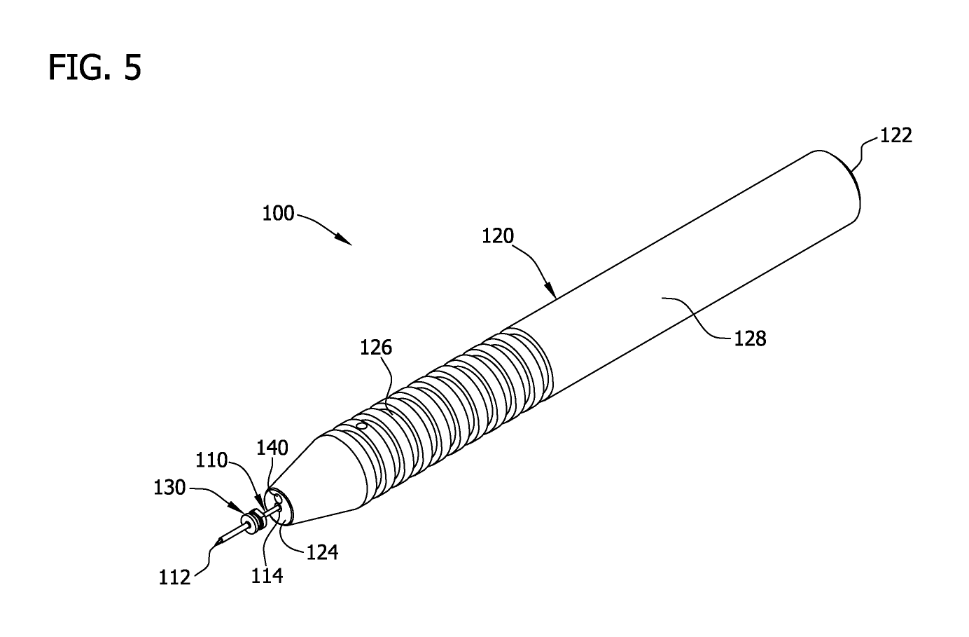

An example trocar system is generally indicated by reference numeral 100 in FIG. 1. The system 100 of this embodiment includes a trocar 110, a handle 120, a cannula 130, and a magnet 140. The trocar 110 has a first end 112 having a sharp point or edge for piercing a portion of a patient's body and forming an incision therein. An opposing second end 114, seen best in FIG. 5, is disposed proximally from the first end 112 of the trocar 110. Typical trocar configurations include hypodermic needles, lancet-shaped needles, stiletto blades, saber-tipped blades, and beveled rods. Furthermore, in some systems the trocar 110 is a blunt insertion device that is used to insert the cannula 130 into an incision formed by another sharp or pointed object.

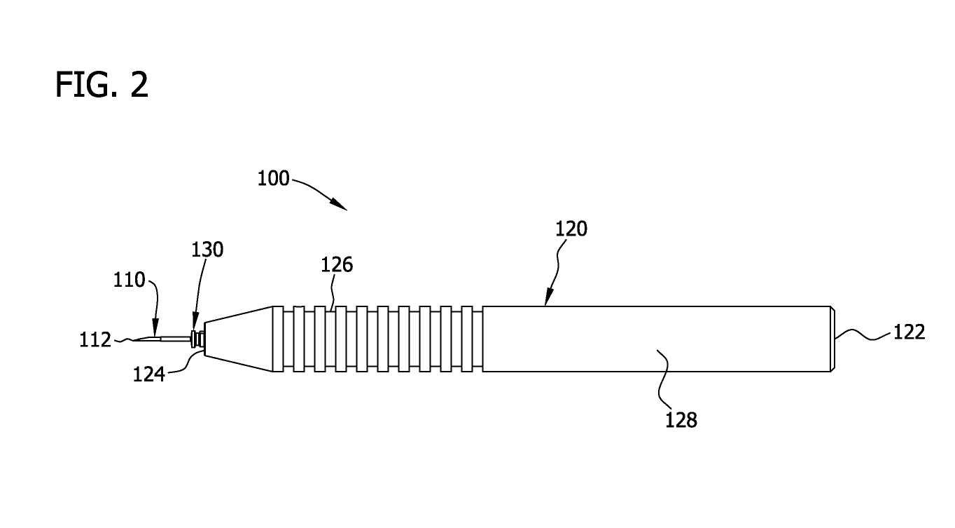

A magnetizable cannula 130 (referred to interchangeably as a “cannula”), as shown in FIGS. 1-3, has a central opening 132 sized to receive the trocar 110. That is, the central opening 132 has a diameter slightly larger than a diameter of the trocar 110. In use, the cannula 130 is positioned within the incision formed by the first end 112 of the trocar 110. The cannula 130 keeps the incision from collapsing or closing after removal of the trocar 110 so that other surgical implements can be inserted through the central opening 132 and into the patient's body. In the example embodiment, the cannula 130 is formed from a ferromagnetic material (e.g., stainless steel) that is attracted to magnets. In other embodiments, the cannula 130 can be formed from any material that is magnetizable (i.e., capable of being attracted to magnets). The positioning of the cannula 130 within the incision and the use of the system 100 generally are described in greater detail below.

As shown in FIG. 1, the handle 120 has a first end 122 and an opposing second end 124 distal from the first end. The second end 124 of the handle 120 is positioned adjacent the second end 114 of the trocar 110 and the two are connected to each other. In the example embodiment, a portion of the trocar 110 adjacent to the second end 114 protrudes into the handle 120 to affect the connection between the two. An adhesive may also be used to attach the trocar 110 to the handle 120.

The handle 120 has a number of circumferential grooves 126 (FIG. 1) formed in an outer surface 128 thereof (only one of which is numbered). The grooves 126 aid a user grasping the handle 120 and their number, shape, and relative position on the handle may be altered without departing from the scope of the embodiments.

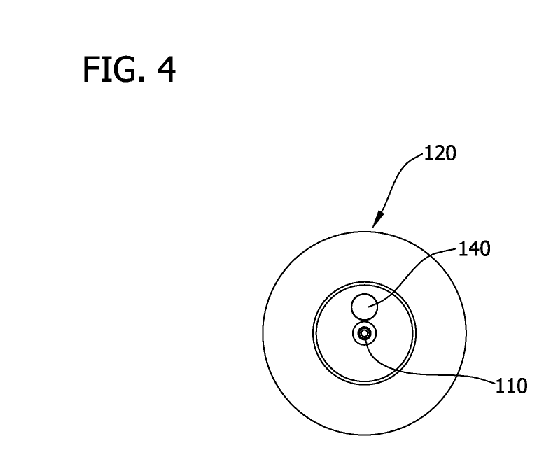

The magnet 140 is positioned adjacent the second end 114 of the trocar 110 and the second end 124 of the handle 120, as shown in FIGS. 1 and 4. Generally, this magnet 140 creates a magnetic field that releasably secures the cannula 130 in a position adjacent the second end 114 of the trocar 110. The cannula 130 is shown in this position in FIG. 1. The strength (i.e., magnitude) of the magnetic field generated by the magnet 140 is such that cannula 130 can be moved away from the second end 114 of the trocar 110 towards the first end 112 (e.g., a position the same or similar to that shown in FIG. 5) of the trocar by a user. The strength of the field is also great enough to releasably secure the cannula 130 in the position shown in FIG. 1 such that prior to its use (i.e., during storage or prior to placement within the incision) the cannula is retained in this position. According to the example embodiment, the strength of the magnetic field generated by the magnet 140 is between about 35 and 50 mega-gauss, although other embodiments may use magnets having field strengths less or greater than this range.

Further, in the example embodiment the magnet 140 is a rare-earth magnet, although in other embodiments it may be made from one or more of gold-plated neodymium, plastic-coated neodymium, or stainless steel. Moreover, the magnet 140 may alternatively be an electromagnet. This electromagnet may be positioned in the handle 120 or be placed adjacent the handle and be selectively energized. When de-energized, the cannula 130 can be readily moved along the trocar 110. When the electro-magnets are energized the cannula 130 is releasably secured in its position adjacent the second end 114 of the trocar 110.

In the example embodiment, a single magnet 140 is used to releasably secure the cannula 130 in the position shown in FIG. 1. As shown in FIGS. 4 and 5, this magnet 140 is positioned within a recess adjacent the second end 124 of the handle 120 and may be secured within the recess by adhesive and/or an interference fit between the magnet and the recess. In the example embodiment, the magnet 140 does not protrude from the second end 124 and is shaped similarly to a rod. In other embodiments, the magnet 140 may instead protrude from the second end 124 of the trocar 120. Moreover, the relative size, shape, and/or placement of the magnet 140 can be altered without departing from the scope of the embodiments. In other embodiments, multiple magnets may be positioned within corresponding multiple recesses formed adjacent the second end 124 of the handle 120. Furthermore, some portion of the handle 120 may be positioned between the magnet 140 and the second end 124 of the handle 120 when the cannula 130 is releasably secured in the position adjacent the second end 114 of the trocar 110. This positioning conceals a portion of the magnet 140 to restrict the magnetic field to an axial direction and decrease radial pull on objects other than the cannula 130.

In different embodiments, the magnet 140 may be a torus-shaped member that has a central opening sized to receive the trocar 110. This magnet can be positioned adjacent the second end 114 of the trocar 110 and/or second end 124 of the handle 120 and secured in this position with adhesive. Such a magnet can also be positioned in a recess formed in the second end 124 of the handle 120. In still other embodiments, the magnet 140 may have a similar or same cross-sectional shape as the trocar 110 or cannula 130 and be positioned between the second end 114 of the trocar 110 and the second end 124 of the handle 120.

In still other embodiments, a portion of the trocar 110 adjacent its second end 114 (i.e., a magnetized portion) can be formed from a material having magnetic properties similar to or the same as the magnet 140. In these embodiments, this magnetized portion of the trocar 110 is in effect a magnet that is integrally formed with the trocar. The magnetized portion creates a magnetic field operable to releasably secure the cannula 130 in a position adjacent the second end 114. Thus a separate magnet as described above is unnecessary.

In operation, the first end 112 of the trocar 110 is used to pierce a portion of a patient's body (e.g., the patient's eye) and form an incision therein. In other embodiments, the piercing action can be performed by a separate sharp or pointed object and the trocar may be a blunt insertion device onto which the cannula is installed.

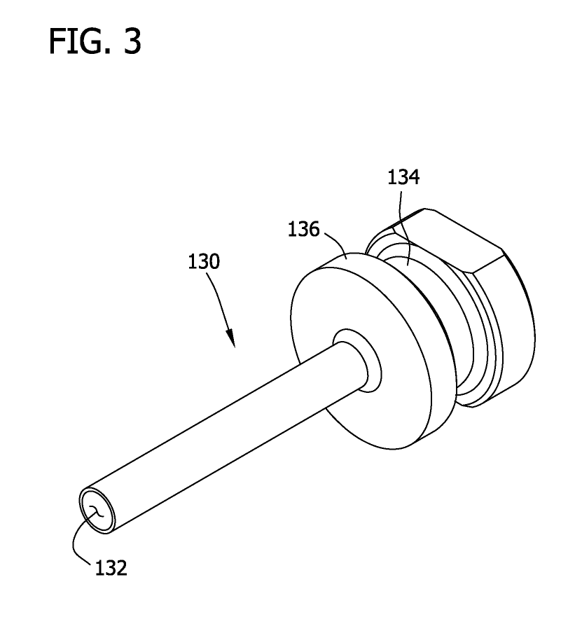

The trocar 110 is then inserted into the patient's body through this incision. The cannula 130 is then typically grasped by a user (e.g., a surgeon, and typically with the aid of a tool such as forceps) and moved from its position adjacent the second end 114 of the trocar 110 towards the first end 112. In the example embodiment, the cannula has a circumferential groove 134 formed in its outer surface 136 (FIG. 3) to aid in the grasping of the cannula by the tool.

To move the cannula 130 from the position shown in FIG. 1, the user must exert sufficient force on the cannula to overcome the force releasably securing the cannula in this position. As described above, this force releasably securing the cannula 130 is resultant from the affects of the magnetic field generated by the magnet 140 on the cannula. The strength of the magnetic field is such that the force is small enough that the user is able to manually (i.e., using the force exerted by a hand and/or tool) overcome it and move the cannula towards the first end 112 of the trocar 110. The size of the magnet 140 and/or material from which it is formed may be selected such that the magnet generates a magnetic field having the desired strength.

In one embodiment, the magnetic field generated by the magnet has a magnitude such that the cannula can be moved away from the second end of the trocar by a user using less than about 0.1 pounds of force, although the force may be less than or greater than this amount in other embodiments. Other design factors and/or variables can affect the amount of force required to move the cannula from the second end of the trocar. Examples include the size of the magnet 140, its position relative to the cannula, materials from which it is formed, procedures by which it is poled, and/or coatings applied to its surface. Moreover, the materials from which the cannula is constructed may be selected such that the magnet generates a magnetic field having the desired strength.

Once positioned near the first end 112 of the trocar 110, the cannula 130 is slid into the incision such that it is positioned between the incision and the trocar. The trocar 110 is then removed from the central opening 132 of the cannula 130 by sliding the trocar in a direction away from the patient's body. The cannula 130 may be grasped while removing the trocar 110 to ensure that the cannula 130 remains in the incision.

Another surgical implement may then be inserted into the central opening 132 of the cannula 130 and used in the surgical procedure. Examples of such surgical implements include fiber optic lights, lasers, probes, infusion systems, or other surgical tools. After completion of the surgical procedure the cannula 130 is removed from the incision.

In other embodiments, the use of a tool to move the cannula 130 along the trocar 110 and insert the cannula in the incision may be unnecessary. In these embodiments, the cannula 130 is inserted into the incision by pushing the trocar 110 into the incision such that the cannula is positioned therein. That is, the cannula 130 remains releasably secured to the trocar 110 adjacent its second end and once the cannula is positioned in the incision, the trocar 110 is removed from the central opening 132. The degree of friction between the cannula 130 and the incision exceeds the force required to detach the cannula from the magnet 140 and withdraw the trocar 110 from the cannula, and thus a tool is not needed to ensure the cannula remains in place in the incision.

Moreover, in still other embodiments a separate sharp or pointed tool is used to form the incision, while a blunt insertion device is used to position the cannula 130 in the incision. In these embodiments, the blunt insertion device has the same functionality as the trocar 110, with the exception for of forming the incision, and thus the blunt insertion device is a type of trocar.

The systems and methods described herein releasably secure the cannula 130 in a position adjacent the second end 114 of the trocar 110. The cannula 130 does not move from this position until a user (e.g., a surgeon) exerts sufficient force on the cannula or on the handle 120 while the contact between the incision and the cannula provides sufficient friction. Accordingly, the cannula 130 is secured in this position during transport and storage of the cannula and prior to its usage without the difficulty and cost of deforming the trocar or the cannula.

Moreover, the embodiments described herein provide the added benefit of releasably securing the cannula 130 to the trocar 110 with a consistent force. Thus, the same amount of force is required to move the cannula 130 along the trocar 110 in similarly configured systems 100. Further, the manufacturing costs and time required to manufacture the system 100 compared to previous retention systems is reduced. The systems 100 described above also increase the visibility of the cannula 130 and the ease with which it can be gripped compared to previous systems which relied on tabs or other extensions on the second end 124 of the handle 120 to retain the cannula.

When introducing elements of the present invention or the embodiments thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the invention, it is intended that all matter contained in the above description and shown in the accompanying figures shall be interpreted as illustrative and not in a limiting sense.

What is claimed is:

1. A trocar system comprising:
a trocar having a first end and a second end, the first end distal from the second end;
a magnet positioned adjacent the second end of the trocar, the magnet generating a magnetic field; and
a magnetizable cannula having a central opening sized to receive the trocar, wherein the magnetic field of the magnet releasably secures the cannula in a position adjacent the second end of the trocar.

2. The system of claim 1 wherein the magnetic field generated by the magnet has a magnitude such that the cannula can be moved away from the second end of the trocar by a user using less than about 0.1 pounds of force.

3. The system of claim 1 further comprising a handle having a first end and a second end, the second end of the handle positioned adjacent the second end of the trocar.

4. The system of claim 3 wherein the magnet is positioned adjacent the second end of the handle.

5. The system of claim 4 wherein the handle has a recess therein adjacent the second end of the handle.

6. The system of claim 5 wherein the magnet is positioned in the recess in the handle.

7. The system of claim 1 wherein the magnet is a torus-shaped member having a central opening sized to receive the trocar.

8. The system of claim 7 wherein the second end of the trocar is received in the central opening of the magnet.

9. A trocar system comprising:
a trocar having a first end and a second end, the first end distal from the second end;
a handle having a first end and a second end, the second end of the trocar connected to the second end of the handle; and
a cannula having a central opening sized to receive the trocar, wherein the cannula is releasably secured in a position adjacent the second end of the trocar by a magnetic field.

10. The system of claim 9 wherein the magnetic field has a magnitude such that the cannula can be moved away from the second of the trocar by a user using less than about 0.1 pounds of force.

11. The system of claim 10 wherein the magnitude of the magnetic field is sufficient to retain the cannula in the position adjacent the second end of the trocar during transport or storage of the system.

12. The system of claim 9 further comprising a magnet positioned adjacent at least one of the second end of the trocar and the second end of the handle.

13. The system of claim 12 wherein the handle has a recess therein adjacent the second end of the handle.

14. The system of claim 13 wherein the magnet is positioned in the recess in the handle.

15. The system of claim 12 wherein the magnet is formed integrally with the trocar and is positioned adjacent the second end of the trocar.

16. The system of claim 12 wherein the magnet is positioned between the second end of the trocar and the second end of the handle.

17. The system of claim 12 wherein the magnet includes at least one of gold-plated neodymium, plastic-coated neodymium, and stainless steel.

18. The system of claim 12 wherein the magnet is one of rod-shaped and torus-shaped.

19. The system of claim 12 wherein at a least a portion of the magnet is concealed by the handle when the cannula is releasably secured in the position adjacent the second end of the trocar to restrict the magnetic field to an axial direction and decrease radial pull on objects other than the cannula.

20. The system of claim 9 wherein the magnetic field is generated by an electro-magnet.

21. A method of using a trocar system, the trocar system comprising a trocar having a first end and a second end, a magnet, and a magnetizable cannula, wherein the magnetizable cannula is releasably secured in a position adjacent the second end of the trocar by a magnetic field of the magnet, the method comprising the steps of;
inserting the first end of the trocar into an incision formed in a patient's body;
inserting the magnetizable cannula into the incision in the patient's body; and
removing the trocar from the magnetizable cannula.

22. The method of claim 21 further comprising inserting a surgical implement through the central opening of the magnetizable cannula and into the patient's body.

23. The method of claim 21 wherein inserting the first end of the trocar into the portion of the patient's body forms an incision in the portion of the patient's body.

24. The method of claim 21 further comprising forming the incision in the patient's body prior to inserting the first end of the trocar into the patient's body.

25. The method of claim 24 wherein the trocar is a blunt insertion device and is inserted through the incision formed in the portion of the patient's body.

26. The method of claim 21 further comprising grasping the magnetizable cannula prior to removing the trocar from the magnetizable cannula.
